(19) United States
(12) Patent Application Publication
(10) Pub. No.: US 2022/0015354 A1
(43) Pub. Date: Jan. 20, 2022

(54) METHOD AND APPARATUS FOR FREEZING OF BIOLOGICAL PRODUCTS

(71) Applicant: VITRAFY LIFE SCIENCES PTY LTD, Hobart (AU)

(72) Inventors: Sean Cameron, Hobart (AU); Brent Owens, Hobart (AU); Brian Taylor, Hobart (AU); Robert Woolley, Hobart (AU); James Groom, Hobart (AU)

(21) Appl. No.: 17/295,555

(22) PCT Filed: Nov. 21, 2019
(86) PCT No.: PCT/AU2019/051279

§ 371 Date: May 20, 2021

(30) Foreign Application Priority Data
Nov. 22, 2018 (AU) 2018904449

(51) Int. Cl. A01N 1/02; F25D 3/10; F25D 11/02

(52) U.S. Cl. CPC A01N 1/0257; A01N 1/0284; F25D 17/02; F25D 3/102; F25D 11/022; A01N 1/0221

(57) ABSTRACT

An apparatus for preserving biological products comprising an inner housing arranged within an outer insulated housing, wherein walls of the inner housing define a compartment for receiving biological products, said walls comprising an inlet wall for inflow of a heat exchange fluid into the compartment, an opposed outlet wall for outflow of a heat exchange fluid out of the compartment, side walls and a base, the side walls and base adjoining the inlet wall to the outlet wall, wherein the inlet wall and outlet wall each include a series of apertures to accommodate a continuous heat exchange fluid flow through the apparatus such that, in operation, biological products received in the compartment of the inner housing are immersed in the heat exchange fluid to exchange heat with the heat exchange fluid.

### FIELD OF THE INVENTION

The present invention relates to methods of freezing biological products and apparatuses for preserving biological products.

### BACKGROUND

The ability to store red blood cells (RBCs) outside of the body has been regarded as a life-saving practice for many years. More recently, the usage of refrigerated stored RBCs in transfusion medicine has been under extensive evaluation. During refrigerated storage RBCs progressively deteriorate and infusion of prolonged stored RBCs has been linked to adverse clinical outcome in terms of postoperative infections, length of hospital stay and mortality.

Concerns regarding the infusion of stored RBCs still remains and a restrictive transfusion strategy is currently being favoured. This has resulted in a revived interest in cryopreservation. Storage of RBCs at ultra-low temperatures halts the cellular metabolism and subsequently prevents the progressive cellular deterioration that has been linked to adverse clinical outcome.

Initially, cryopreservation appeared a promising approach for maintaining RBCs viable for prolonged periods of time. However, the clinical applicability of cryopreserved RBCs (commonly known as “frozen RBCs”) was hampered by the expensive, time-consuming and inefficient nature of this preservation method.

Requirements of Refrigerated Stored RBCs

Currently RBCs are routinely stored at 2-6° C. for a maximum of 5 to 6 weeks, depending on the preservation solution used. Cryopreservation, on the other hand, enables storage of RBCs for years. Cryopreservation is currently a valuable approach for long-term storage of RBCs from donors with rare blood groups and for military deployment. However, stockpiling cryopreserved RBCs can also be beneficial in emergency or clinical situations, where the demand exceeds the supply of RBCs. The shelf life of cryopreserved RBCs using current methods is up to ten years.

International guidelines require that haemolysis in a refrigerated RBC storage unit must remain below allowable levels (i.e., 0.8% in Europe and 1% in The United States) and that at least 75% of the infused RBCs must still circulate 24 hours after infusion.

However, the guidelines do not specifically reflect the RBCs' ability to function after infusion.

Quality of Stored RBCs

Although storage at 4° C. slows down the biochemical processes in the RBCs, cellular metabolism is not completely suppressed at these temperatures. During refrigerated storage a variety of changes have been observed that could compromise the RBCs' ability to function after infusion. These changes include decreased concentrations of 2,3-diphosphoglycerate (DPG), adenosine triphosphate (ATP) and membrane sialic acid content. Other changes include translocation of phosphatidylserine (PS) to the cell surface, oxidative injury to membrane lipids and proteins, shape change to spheroechinocytes, membrane blebbing and accumulation of potassium, free haemoglobin (Hb), cytokines, bioactive lipids and (pro-coagulant) microvesicles in the RBC storage unit.

The RBCs' rheologic properties also become impaired during refrigerated storage. Refrigerated RBCs demonstrate an increased tendency to aggregate and adhesion to endothelial cells (ECs), as well as reduced deformability from the second week of storage. These changes may hamper the RBCs' ability to function properly in the microcirculation.

Storage of RBCs at ultra-low temperatures ceases the biological activity of RBCs, enabling them to be preserved for prolonged periods of time. In general, either high concentrations of cryoprotective additives or rapid freezing rates are necessary to prevent cell damage. At slow cooling rates, extra-cellular ice formation will occur. As ice forms, the solute content of the unfrozen fraction becomes more concentrated. The resulting osmotic imbalance causes fluid to move out of the RBC and intracellular dehydration occurs. On the other hand, at rapid cooling rates the RBC cytoplasm becomes super-cooled and intracellular ice formation occurs, which subsequently can lead to mechanical damage.

In order to minimise freezing damage, cryoprotective additives are crucial. Over the years, different non-permeating and permeating additives for the cryopreservation of RBCs have been investigated. Non-permeating additives such as hydroxyethyl starch and polyvinylpyrrolidone, as well as a variety of glycols and sugars appeared promising because it was proposed that removal from thawed RBCs prior to transfusion was not required.

Conversely, the permeating additive glycerol is known for its ability to protect RBCs at ultra-low temperatures. The concentration of glycerol that is necessary to protect the RBCs is dependent on the cooling rate and the storage temperature. Glycerol protects the RBCs by slowing the rate and extent of ice formation while minimising cellular dehydration and solute effects during freezing.

Requirements of Cryopreserved RBCs

Although preservation of RBCs at ultra-low subzero temperatures enables them to be preserved for years, once thawed, the shelf life of RBCs is limited. Deglycerolised RBCs are primarily stored in saline-adenine-glucose-mannitol (SAGM) preservation solution for up to 48 hours or in AS-3 preservation solution for up to 14 days. Cryopreserved RBCs need to be deglycerolised to reduce the residual glycerol content to below 1%. Furthermore, the RBCs are subject to the abovementioned international guidelines requiring that haemolysis in the RBC units must remain below allowable levels (i.e. 0.8% in Europe and 1% in The United States) and that the RBC post-thaw recovery after deglycerolisation (i.e. freeze-thaw-wash recovery) must exceed 80%. Also, at least 75% of cryopreserved RBCs must still circulate 24 hours after infusion.

Freezing Methods with Glycerol

Currently there are two freezing methods accepted for the preservation of RBCs with glycerol.

1. RBCs can be frozen rapidly in liquid nitrogen using a low-glycerol method (LGM) with a final concentration of approximately 20% glycerol (wt/vol) at temperatures below −140° C.
2. RBCs can be frozen slowly using a high-glycerol method (HGM), allowing storage of RBC units with a final concentration of approximately 40% (wt/vol) glycerol at temperatures between −65° C. and −80° C.

Cryopreserved RBCs are less efficient due to the cellular losses that occur during the processing procedure. This cell loss is more pronounced in HGM cryopreserved RBCs (approximately 10-20%) since these RBCs require more extensive washing. However, despite the higher yield of RBCs with the LGM, it is generally considered that HGM cryopreserved RBCs can tolerate wide fluctuations in temperature during freezing and are more stable during post-thaw storage. In addition, HGM cryopreserved RBCs do not require liquid nitrogen which eased storage and transportation conditions. Consequently, the HGM is currently the most applicable RBC freezing method in Europe and the United States.

The storage method associated with the HGM of cryopreservation results in intracellular dehydration due to the high glycerol content and storage temperature ranges. Preferred embodiments of the present invention seek to utilise lower glycerol content, thereby minimising cellular dehydration and solute effects, while extending the shelf life of cryopreserved RBCs.

### SUMMARY

As used herein, “biological products” (or “biological materials”) includes the following non-exhaustive list of materials: blood, plasma, platelets, leucocytes or other blood products; germs, bacteria, fungi, or other microorganisms; organs, seminal fluid, eggs, colostrum, skin, serum, vaccines, stem cells (eg from bone marrow, umbilical cord blood, amniotic fluid, etc), umbilical cords, bone marrow, germ cells, tumour cells, colostrum, vaccines, and plant cells.

According to a first aspect of the present invention, there is provided an apparatus for preserving biological products comprising an inner housing arranged within an outer insulated housing, wherein walls of the inner housing define a compartment for receiving biological products, said walls comprising an inlet wall for inflow of a heat exchange fluid into the compartment, an opposed outlet wall for outflow of a heat exchange fluid out of the compartment, side walls and a base, the side walls and base adjoining the inlet wall to the outlet wall, wherein the inlet wall and outlet wall each include a series of apertures to accommodate a continuous heat exchange fluid flow through the apparatus such that, in operation, biological products received in the compartment of the inner housing are immersed in the heat exchange fluid to exchange heat with the heat exchange fluid.

According to a second aspect of the present invention, there is provided method of determining an amount of cryoprotectant to be added to a biological product prior to preservation, comprising:
a. determining the total surface area of an approximated geometry of the biological product, including an initial amount of cryoprotectant, to be preserved, wherein the biological product, cryoprotectant and any packaging define a sample;
b. estimating thermal properties of the sample;
c. performing computational fluid dynamics analysis on the sample within the apparatus of any one of the preceding claims based on flow constraints including any one or more of: an approximated geometry of the sample; thermal properties of the sample; the apparatus geometry; predetermined arrangement of sample in the apparatus; a predetermined inlet temperature of heat exchange fluid; and a predetermined increase in temperature of the heat exchange fluid from inlet to outlet;
d. determining an average temperature reduction rate of the core of the sample at a predetermined sample surface temperature and corresponding heat exchange fluid flow rate to obtain the average temperature reduction rate; and
e. if the fluid flow rate calculated at step (d) corresponds to a pump duty of the apparatus that is below a predetermined pump duty, selecting an amount of cryoprotectant that is a predetermined amount less than the initial amount to define a new initial amount and, if the fluid flow rate calculated at step (d) corresponds to a pump duty that is equal to a predetermined pump duty, selecting the initial amount of cryoprotectant as said amount of cryoprotectant to be added to a biological product prior to preservation; and
f. if the fluid flow rate calculated at step (d) corresponds to a pump duty that is below a predetermined pump duty, repeating steps (a) to (e) until the fluid flow rate calculated at step (d) corresponds to a pump duty that is equal to a predetermined pump duty.

According to a third aspect of the present invention, there is provided method of determining an amount of cryoprotectant to be added to a biological product prior to preservation, comprising:
a. determining the total surface area of an approximated geometry of the biological product, including an initial amount of cryoprotectant, to be preserved, wherein the biological product, cryoprotectant and any packaging define a sample;
b. estimating thermal properties of the sample;
c. performing computational fluid dynamics analysis on the sample within the apparatus of any one of claims 1 to 8 based on flow constraints including any one or more of: an approximated geometry of the sample; thermal properties of the sample; the apparatus geometry; predetermined arrangement of sample in the apparatus; and a predetermined increase in temperature of the heat exchange fluid from inlet to outlet;
d. determining an average temperature reduction rate of the core of the sample at a predetermined sample surface temperature and corresponding inlet temperature of heat exchange fluid to obtain the average temperature reduction rate, and
e. if the inlet temperature of heat exchange fluid determined at step (d) corresponds to an evaporator duty that is below a predetermined evaporator duty, selecting an amount of cryoprotectant that is a predetermined amount less than the initial amount to define a new initial amount, and, if the fluid flow rate calculated at step (d) corresponds to a pump duty that is equal to a predetermined pump duty, selecting the initial amount of cryoprotectant as said amount of cryoprotectant to be added to a biological product prior to preservation; and
f. if the fluid flow rate calculated at step (d) corresponds to an evaporator duty that is below a predetermined evaporator duty, repeating steps (a) to (e) until the fluid flow rate calculated at step (d) corresponds to a pump duty that is equal to a predetermined pump duty.

According to a fourth aspect of the present invention, there is provided use of the apparatus of the first aspect to preserve a biological product.

The apparatus may include trays, racks or baskets designed to hold the relevant biological product to be preserved.

### BRIEF DESCRIPTION

Embodiments of the present invention will now be described, by way of non-limiting example only, with reference to the accompanying drawings, in which:

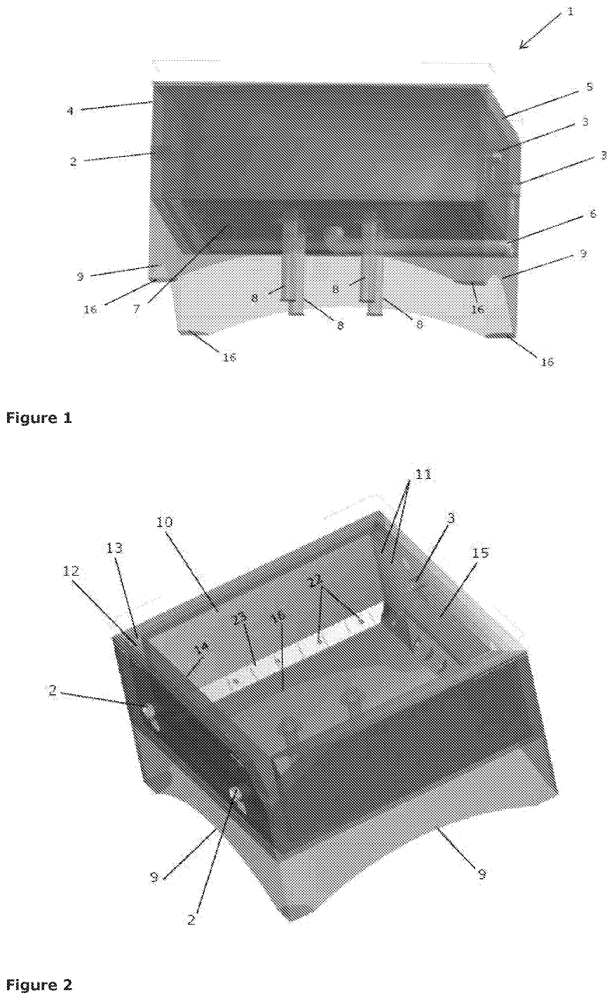

FIG. 1 is a lower perspective view of a tank for preservation of biological products;

FIG. 2 is an upper perspective view of a tank for preservation of biological products;

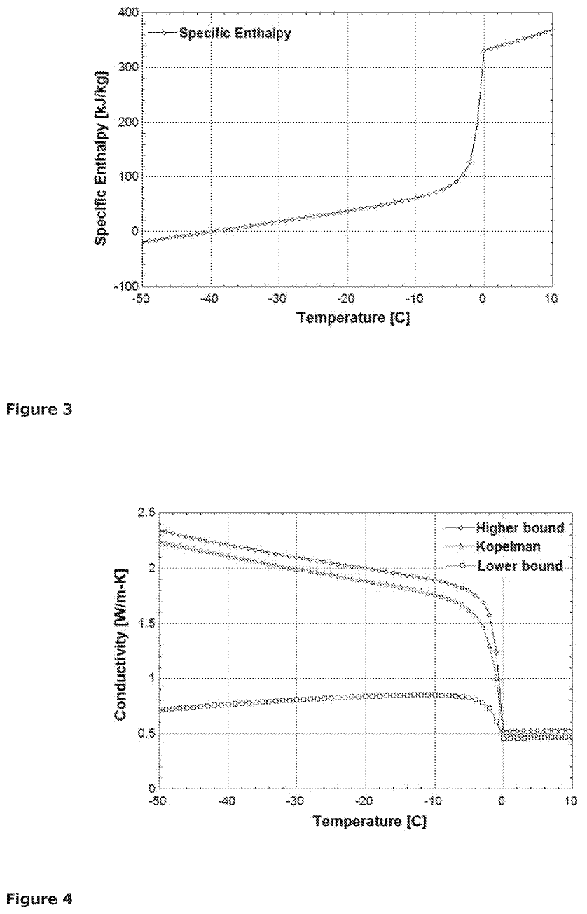

FIG. 3 is a graph showing the specific enthalpy of blood at various temperatures;

FIG. 4 is a graph showing the conductivity of blood at various temperatures;

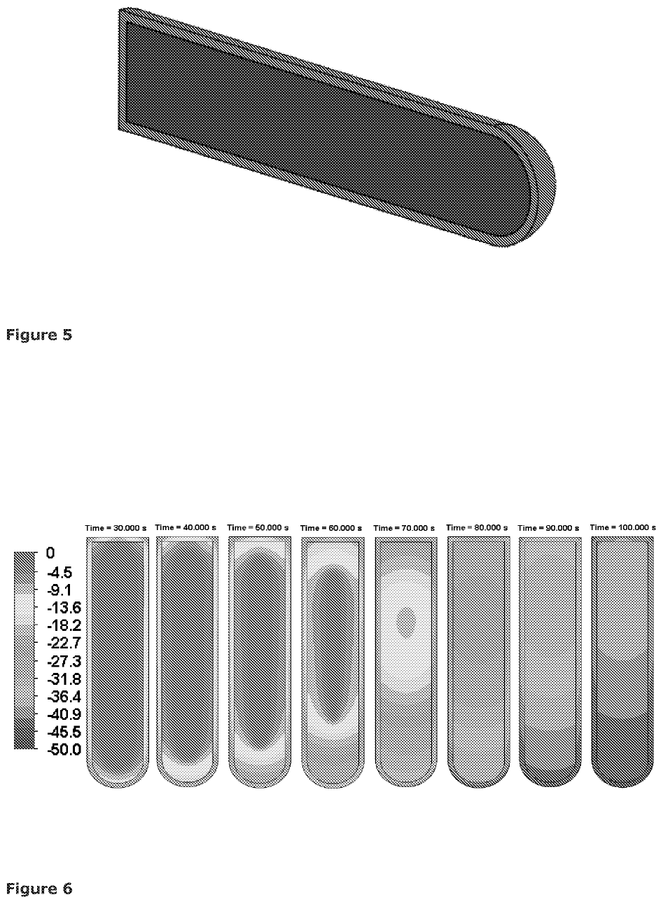

FIG. 5 is a representation of a cryovial of blood;

FIG. 6 shows temperature plots of blood at various time intervals;

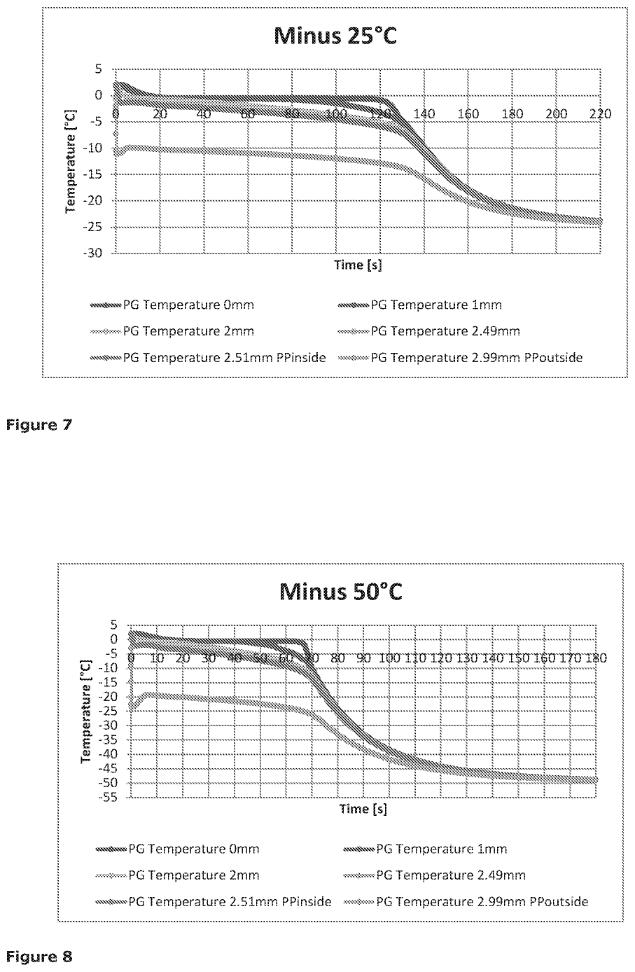

FIG. 7 shows the temperature/time profiles of geometric increments of blood in a polypropylene-walled cryovial subjected to cryopreservation with a heat exchange fluid inlet temperature of −25° C.;

FIG. 8 shows the temperature/time profiles of geometric increments of blood subjected to cryopreservation with a heat exchange fluid inlet temperature of −50° C.;

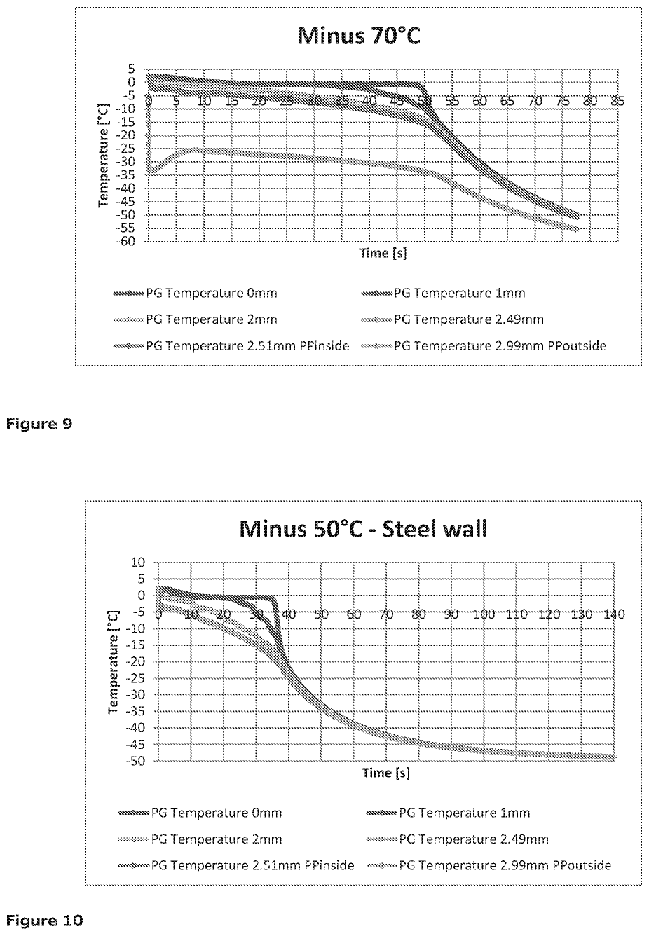

FIG. 9 shows the temperature/time profiles of geometric increments of blood subjected to cryopreservation with a heat exchange fluid inlet temperature of −70° C.;

FIG. 10 shows the temperature/time profiles of geometric increments of blood in a steel-walled cryovial subjected to cryopreservation with a heat exchange fluid inlet temperature of −50° C.;

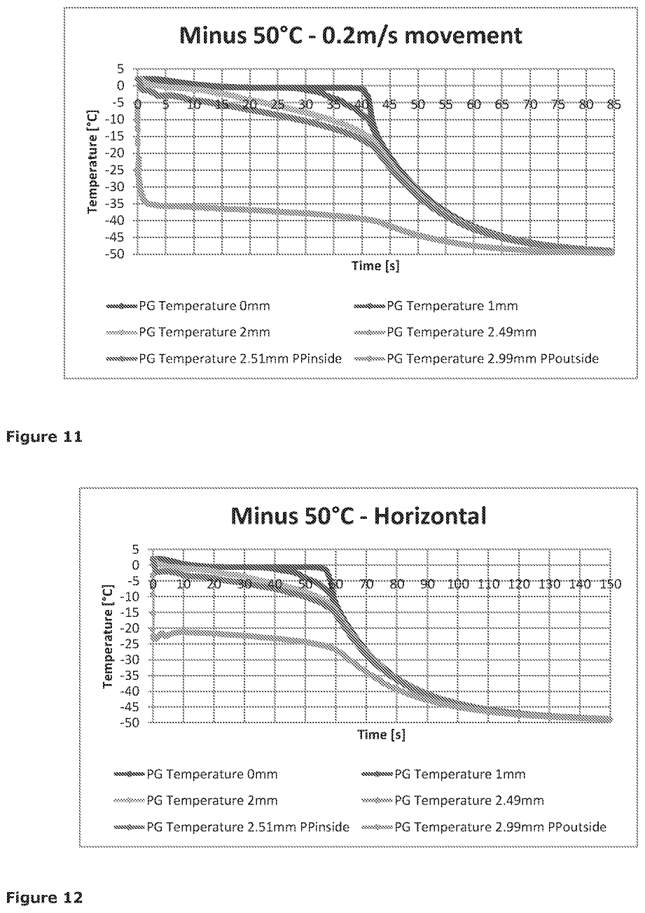

FIG. 11 shows the temperature/time profiles of geometric increments of blood subjected to cryopreservation with a heat exchange fluid inlet temperature of −50° C. and relative motion between the cryovial and heat exchange fluid of 0.2 m/s;

FIG. 12 shows the temperature/time profiles of geometric increments of blood subjected to cryopreservation with a heat exchange fluid inlet temperature of −50° C. and horizontal orientation of the cryovial;

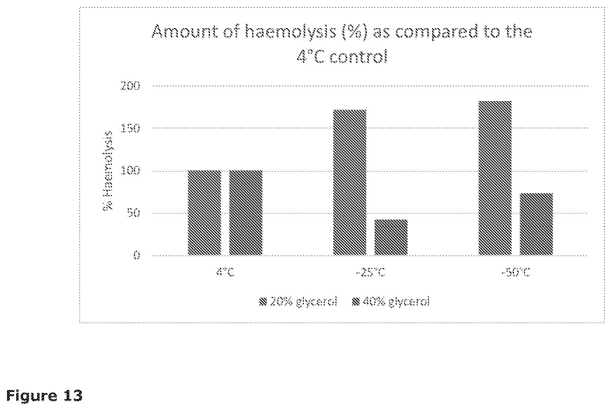

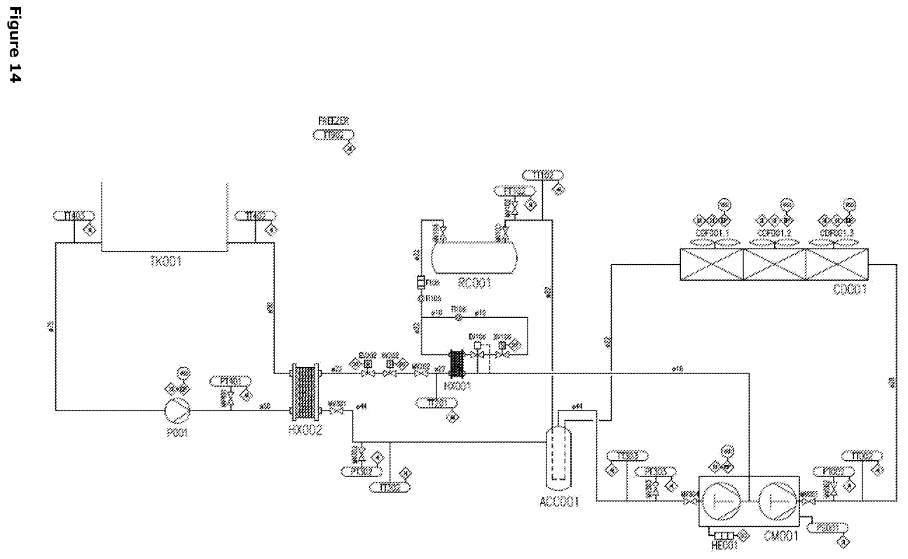

FIG. 13 is a graph showing percentage haemolysis for different preservation scenarios; and

FIG. 14 is a piping and instrumentation diagram of the refrigeration system.

### DETAILED DESCRIPTION

FIGS. 1 and 2 show an immersion tank 1 for preservation of a biological product (or biological material). The tank 1 is constructed of steel to conform with ASTM A240. The tank 1 has two heat exchange fluid inlets 2 and two heat exchange fluid outlets 3, the inlets 2 being situated on an inlet wall 4 and the outlets 3 being situated on an outlet wall 5.

FIG. 2 shows an inner housing 10 situated internally of the outer walls of the tank 1. Inner housing 10 has an inlet wall 14, outlet wall 15 and base 16, each including apertures 11 to allow inflow and outflow of heat exchange fluid into and out of the inner housing 10. The apertures 11 are provided in four rows of ten on the inlet wall 14 and outlet wall 15, and ten rows of ten on the base 16. The apertures 11 on inlet wall 14 and base 16 are 10 mm in diameter and the apertures 11 on the outlet wall 15 are 20 mm in diameter. The inlet wall 14 is spaced 100 mm away from an inner face 12 of the inlet wall 2, thus providing void 13. A similar void is provided between outlet wall 15 and an inner face of outlet wall 5.

A 100 mm void space is further provided in the base. Inner face 12 is defined by steel sheet formwork arranged 50 mm from the inlet wall 2 and secured by brackets, providing a cavity into which polyurethane foam insulation is pumped during manufacture of the tank 1. Insulation is provided in a similar manner in all four walls of the tank from the top of the tank to approximately 595 mm down the walls of the tank.

Rows of holes 22 of 30 mm diameter are provided along strips 23 which sit at an angle of approximately 45° between the base 7 and the walls of the tank along the bottom of each wall. The strips 23 are provided to brace the tank structure and can also be used as guides to prevent the trays or basket resting against the walls or base of the inner housing 10. It will be appreciated that other arrangements are possible which also brace the tank structure and perform a guide function. The holes 22 help to reduce stagnation of the heat exchange fluid that may accumulate in these regions of the tank due to the presence of the strips 23.

A drain 6 is provided from the base 7 of the tank 1 and is shaped as an elbow pipe directed to extend beyond the outlet wall 5 of the tank 1, below the heat exchange fluid outlets 3. The heat exchange fluid inlets 2 and the heat exchange outlets 3 have a diameter of 80 mm.

The tank 1 further includes a lid formed of steel sheet (not shown). The base 7 of the tank 1 includes four central leg portions 8 supporting the central weight of the tank 1, as well as feet 16 situated at the corners of the tank 1 and formed at the ends of the tank walls. Cut-out portions 9 are provided on the lower ends of the tank walls to provide access for maintenance of the base 7 of the tank. The tank 1 has a height of about 1.105 m and is arranged in a square configuration having side lengths of 1.705 m.

In use, the tank 1 is filled with heat exchange fluid which does not freeze above −70° C. The heat exchange fluid is pumped into the tank 1 via the heat exchange fluid inlets 2 into cavity 13 at a volumetric flow rate of 17 cubic metres per hour. Pressure is built up in the cavity 13 as heat exchange fluid is forced through the restricted areas of the apertures 11, thus reducing the volumetric flow rate but increasing velocity of the fluid entering the inner housing 10. Some fluid will also travel below the inner housing 10 and be forced up to the opposing cavity in the outlet wall 5, with some fluid also travelling up through apertures 11 provided in the base 7 of the inner housing 10. The apertures 11 provide improved distribution of cold fluid to all parts of the tank and minimise the occurrence of hot spots which would otherwise be likely to occur away from the inlet area. As the heat transfer fluid flows continuously through the tank 1, heat is removed from the biological product, and the heated heat exchange fluid leaving the tank 1 will then be exchanged with a refrigeration system which continuously cools the heat exchange fluid. The heat exchange fluid itself exchanges heat with refrigerant in the refrigeration system.

Preferably, a low range heat transfer fluid is used as the immersion fluid for the tank which, advantageously, has a relatively low viscosity even at very low temperatures, thus reducing the pump power requirements for the system. The below table (Table 1) specifies some of the thermal properties of the heat transfer fluid.

TABLE 1
Thermal properties of the heat transfer fluid

| Temperature | Density | Specific Heat | Conductivity | Viscosity |
|---|---|---|---|---|
| [° C.] | [kg/m3] | [J/kg-K] | [W/m-K] | [Pa-s] |
| −73.3 | 827 | 1630 | 0.1591 | 0.154 |
| −59 | 820 | 1717 | 0.1585 | 0.038 |
| −45.6 | 815 | 1760 | 0.1574 | 0.016 |
| −17.8 | 791 | 1840 | 0.1539 | 0.005 |
| 23.9 | 755 | 2010 | 0.1504 | 0.0015 |

At each of the above temperatures, the heat exchange fluid has a density that is very low and less than that of water. Advantageously, if any breakage or spillage were to occur during operation of the tank, the broken or spilled matter will tend to sink to a lower portion of the tank, facilitating drainage of that matter without substantial loss of heat exchange fluid. It will be appreciated that any suitable heat exchange fluid can be used, provided that it has a low enough viscosity that it will not require excessive pump power at the required low temperatures for preservation. It is also preferable that the heat exchange fluid be safe for biological products.

TABLE 2
Flow rate of heat transfer fluid for various temperature
differences @ 20 kW (−50° C.)

| Temperature difference | Mass flow rate | Volumetric flow rate |
|---|---|---|
| [° C.] | [kg/s] | [m³/hr] |
| 1 | 11.5 | 50.9 |
| 2 | 5.8 | 25.5 |
| 3 | 3.9 | 17 |
| 4 | 2.9 | 12.7 |
| 5 | 2.3 | 10.1 |
| 10 | 1.15 | 5.1 |

Table 2 above provides the temperature difference between the tank inlet and outlet for various flow rates of heat exchange fluid, assuming 20 kW of heat is extracted from the fluid in the tank. From Table 2, it can be seen that a temperature difference of 3° C. between inlet and outlet can be achieved using a mass flow rate of approximately 4 kg/s. This temperature difference was deemed an acceptable temperature rise in terms of evaporator duty required as well as cooling of the product required. The acceptable temperature rise must be balanced against costs associated with the maximum number of product that can be processed at once to make the system economically viable. It will be appreciated that a higher flow rate may be desirable in increasing the heat transfer between the heat exchange fluid and the consumable product. However, a higher flow rate will also cause higher flow resistance and thus a higher pumping power would be required.

The inventors have found that by using an increased flow rate of heat exchange fluid to rapidly reduce the temperature of the biological product, the biological product can be preserved with a reduced level of cryoprotectant while minimising damage due to any ice crystal formation that would ordinarily occur during the freezing process.

The method of determining the correct temperature and velocity to achieve vitrification of RBCs and other biological material is based on calculations including the thermal properties, surface area and product load volumes. The calculated heat transfer coefficients then inform final apparatus operating conditions enabling the cryopreservation to occur.

In an example, the freezing of 0.5 mL blood in a cryovial is investigated.

FIG. 5 shows a CAD model of the cryovial made of polypropylene, 6 mm outer diameter, 27.5 mm length, 0.5 mm wall thickness having a 0.5 mL internal volume.

Computational Fluid Dynamics (CFD) is used to calculate the freezing times for a cryovial filled with blood. For the CFD analysis, it is assumed that the cryovial has a uniform temperature of 2° C. at the start of the simulation. It is immersed in a heat transfer fluid at temperatures of minus 25, minus 50 and minus 70° C. respectively. The transient CFD then automatically calculates the heat transfer between the heat transfer fluid and the external surface of the cryovial. The CFD also calculates the conduction of heat throughout the cryovial (blood and polypropylene).

Movement of blood during freezing is ignored, i.e. the blood is assumed to be ‘solid’. The thermal properties of blood were based on assuming it consists of 85% water and 15% protein. Thermal properties of biological products can be obtained through methods known to those skilled in the art, or looked up in thermal property tables known to those skilled in the art.

FIGS. 3 and 4 show respectively the specific enthalpy and thermal conductivities as calculated for blood. The conductivity is calculated by the Kopelman method.

The table below gives the freezing times of the core for temperatures of the heat transfer fluid of minus 25, minus 50 and minus 70° C. respectively.

TABLE 3
Freezing time results

| | Heat | Time for | Time for | Time for | Time for |
|---|---|---|---|---|---|
| | transfer | core to | core to | core to | core to |
| | fluid | start | reach | reach | reach |
| | Temperature | freezing | minus 30° C. | minus 40° C. | minus 50° C. |
| # | [° C.] | [seconds] | [seconds] | [seconds] | [seconds] |
| 1 | −25 | 123 | ~220 | N/A | N/A |
| 2 | −50 | 66 | 86 | 105 | ~180 |
| 3 | −70 | 48 | 60 | 67 | 78 |

The temperature/time plots corresponding to each of the scenarios in Table 3 are shown in FIGS. 7 to 9. FIG. 6 shows temperature plots for the minus 50° C. temperature case for the heat transfer fluid at 10 second intervals, from 30 to 100 seconds.

Referring to FIG. 7, the core temperature starts at approximately 2° C. at time 0 s and is cooled to a temperature of approximately −24° C. by 220 s, resulting in an overall average temperature reduction rate of approximately 7° C. per minute. However, observing the window of time between approximately 125 s and 150 s, a rapid decrease in core temperature of approximately 14° C. occurs, resulting in an average temperature reduction rate over that period of time of about 34° C. per minute.

Referring to FIG. 8, the core temperature starts at approximately 2° C. at time 0 s and is cooled to a temperature of approximately −49° C. by 180 s, resulting in an overall average temperature reduction rate of approximately 17° C. per minute. However, observing the window of time between approximately 69 s and 75 s, a rapid decrease in core temperature of approximately 14° C. occurs, resulting in an average temperature reduction rate over that period of time of about 140° C. per minute.

Referring to FIG. 9, the core temperature starts at approximately 2° C. at time 0 s and is cooled to a temperature of approximately −50° C. by 77.5 s, resulting in an overall average temperature reduction rate of approximately 40° C. per minute. However, observing the window of time between approximately 49 s and 52.5 s, a rapid decrease in core temperature of approximately 14° C. occurs, resulting in an average temperature reduction rate over that period of time of 240° C. per minute.

Advantageously, the rapid temperature reduction may take place between approximately −2° C. and −15° C., which is considered to be an important range for supercooling of many biological products. In the above examples, the window of rapid temperature reduction approximately coincides with this temperature range.

In the above examples, a steepest section of the graph is visually assessed to determine the approximated maximum rate of change of temperature. Another option is to model the data and plot the derivative, thus determining the highest instantaneous rate of change of the product. The required rate of change can be stipulated as being at least above a given rate of reduction over a given period of time. For example, it may be necessary for the temperature reduction rate to be greater than 100° C. per minute for a period of 20 s.

The following additional scenarios were performed to investigate the effect of alternative scenarios. All of the following analyses were performed with a minus 50° C. temperature of the heat transfer fluid. These included:
a. The effect of the polypropylene wall: polypropylene has a low thermal conductivity and thus a high resistance for heat to flow from the blood to the heat transfer fluid. In this case the polypropylene was replaced with a steel wall. This demonstrates the effect of using a higher conductivity material and/or a thinner wall thickness.
b. The effect of moving the cryovial horizontally through the heat transfer fluid at 0.2 m/s. Movement of the cryovial (or the heat transfer fluid) will enhance the heat transfer between the cryovial surface and the heat transfer fluid.
c. The effect of orientating the cryovial horizontally. During the freezing process the cryovial gives off heat to the heat transfer fluid. Hot fluid rises, thus the warmer heat transfer fluid next to the cryovial rises, creating some movement of the heat transfer fluid that increases heat transfer from the cryovial surface. Orientating the cryovial horizontally may have an effect on the surface heat transfer.

The table below shows the freezing times for the alternative scenarios described above. The results of scenario 2 as described in Table 3 are repeated for ease of reference. FIGS. 10 to 12 show respectively the corresponding temperature/time profiles.

TABLE 4
Freezing time results - alternative scenarios

| | | Time for | Time for | Time for |
|---|---|---|---|---|
| | | core to | core to | core to |
| | | start | reach | reach |
| | | freezing | minus 30° C. | minus 40° C. |
| # | Description | [seconds] | [seconds] | [seconds] |
| 2 | i.e. #2 given | 66 | 86 | 105 |
| | in Table 3 | | | |
| 4 | Steel Wall | 35 | 46 | 64 |
| 5 | 0.2 m/s movement | 40 | 49 | 85 |
| 6 | Horizontal | 56 | 73 | 88 |

These results show that using a higher conductivity/lower thickness cryovial material can have a significant effect. Movement of the heat transfer fluid/cryovial thus increases the surface heat transfer, resulting in an effect on freezing times. Orientating the cryovial horizontally also has a positive effect.

Testing of the RBCs after being subjected to the present cryopreservation method was undertaken.

Approximately 30 ml (6×5 mls) of blood was obtained from a healthy volunteer by venepuncture. Whole blood was centrifuged for 12 minutes at 1100×g at 4° C. The buffy coat and most of the plasma was removed and discarded. The concentrated RBCs were then washed twice with Phosphate Buffered Saline (PBS; pH 7.4), and resuspended in PBS to a final haematocrit (Hct) value of 50±10%. The RBC concentrates were then transferred to 50 ml plastic Falcon tubes for glycerolization.

Glycerolisation

RBCs were glycerolized at room temperature to obtain a final concentration of 20% or 40% glycerol.

The six combinations were assayed (each in triplicate):

| | | |
|---|---|---|
| RBC concentrate (stored | 20% Glycerol | 40% Glycerol |
| at 4° C.) | | |
| RBC concentrate (preserved | 20% Glycerol | 40% Glycerol |
| at −25° C.) | | |
| RBC concentrate (preserved | 20% Glycerol | 40% Glycerol |
| at −50° C.) | | |

An equal amount of standard 57% (wt/vol) glycerol was added to the RBC concentrate to achieve a final concentration of approximately 40% (wt/vol) glycerol. A 25% (wt/vol) glycerol mixture was added in a ratio of 5:1 to the RBCs to achieve a final concentration of approximately 20% (wt/vol) glycerol. The ultimate solution was subjected to the cryopreservation process, thawed and held overnight at 4° C. Control RBC concentrates (not subjected to the cryopreservation process) were also held at 4° C. overnight.

Deglycerolization

RBC concentrates were equilibrated at room temperature for 30 minutes with gentle inversion. The suspension was then centrifuged for 12 minutes at 1100×g and the supernatant discarded. The RBC suspensions (0.5 mls) were deglycerolized by repeated washing with NaCl solutions of decreasing osmolality as follows:
0.125 ml of 8% (12% for the 40% glycerol sample) NaCl, incubated for 3 minutes at room temp
0.625 ml of 0.9% NaCl, incubated for 3 minutes at room temp
0.75 ml of 0.9% NaCl, incubated for 3 minutes at room temp
4 ml of 0.9% NaCl, incubated for 3 minutes at room temp

The suspension was centrifuged for 12 minutes at 1100×g and the supernatant was discarded. The RBCs were resuspended in 1 ml of PBS.

Evaluation of Haemolysis

Deglycerolised cells (described above) were stored in PBS at 4° C. for 2 hours, after which haemolysis was measured. Cells were centrifuged for 1 minute at 2860×g to separate RBC from the supernatant. Supernatant was transferred to a plastic curvette (1 cm) and the fraction of free haemoglobin was determined by measuring the absorbance at 540 nm in a spectrophotometer.

Results:

The raw data for each treatment is given in the table below. The amount of free haemoglobin correlates to the amount of lysed RBCs. Both methods resulted in low levels of haemolysis.

| | |
|---|---|
| Replicate | Absorbance at 540 nm |

| Temp | (n = 3) | 20% glycerol | 40% glycerol |
|---|---|---|---|
| 4° C. | Replicate 1 | 0.020 | 0.159 |
| | Replicate 2 | 0.012 | 0.033 |
| | Replicate 3 | 0.018 | 0.099 |
| | Mean | 0.017 | 0.097 |
| | Std dev | 0.003 | 0.051 |
| −25° C. | Replicate 1 | 0.033 | 0.032 |
| | Replicate 2 | 0.023 | 0.022 |
| | Replicate 3 | 0.030 | 0.068 |
| | Mean | 0.029 | 0.041 |
| | Std dev | 0.004 | 0.020 |
| −50° C. | Replicate 1 | 0.040 | 0.073 |
| | Replicate 2 | 0.020 | 0.032 |
| | Replicate 3 | 0.031 | 0.107 |
| | Mean | 0.030 | 0.071 |
| | Std dev | 0.008 | 0.031 |

FIG. 13 shows the amount of haemolysis in each of the above scenarios compared with the control.

The results show that the cryopreservation procedure described herein protected RBCs from degradation when 40% glycerol was used as a cryoprotectant. At both temperatures (−25° C. and −50° C.) tested, the process was superior to the control. It is considered that with optimisation of the system and process, the 20% glycerol case would at least show equivalence with the control.

Determination of Required Cryoprotectant Amount

Analysis is performed by investigating the influence of varying input parameters of the preservation system. This may include the geometry of the product, the starting temperature of the product, the characteristics of the packaging and the characteristics of the racking systems utilised. The method involves dividing the biological product into geometrical increments (e.g. cylindrical shells for bottles or test tubes). For every one of these increments, a conservation of energy equation is solved, i.e. for a given time-step, a certain amount of energy is removed from a shell, resulting in a decrease in temperature of that shell. The amount of energy removed is a function of the temperatures of the adjacent shells, as well as the resistance to heat flow between the shells. This involves taking into account thermal properties of the biological product as a function of temperature.

Analysis is performed assuming that the blood can be treated as a solid mass having a starting temperature of 2° C. and thermal properties which can be identified, estimated or calculated using methods that will be known to the person skilled in the art.

On the basis of the total surface area of the product, load volume of the product in the tank, a pre-selected inlet temperature of heat exchange fluid, a pre-selected acceptable outlet temperature of heat exchange fluid (e.g. 3° C. greater than the inlet temperature), the thermal properties of the product (including cryoprotectant) and packaging and pre-selected velocity of fluid through the tank, the rate of temperature reduction of product can be simulated as detailed above.

The rate of temperature reduction within a given snapshot of time may be, for example, 90° C. per minute or more. If the resultant velocity and temperature reduction are acceptable from a practical standpoint (e.g. if the pump duty is acceptable based on the viscosity of heat exchange fluid at the selected temperature or if the evaporator duty is acceptable based on the required heat removal), a higher temperature reduction rate can be selected with a correspondingly lower amount of cryoprotectant in the product (such that preservation can still occur without damage to the product). The newly selected temperature reduction rate, and thus higher fluid velocity, can then be simulated to determine whether they are acceptable from a practical standpoint, as detailed above.

Once the highest practical temperature reduction rate is determined (and correspondingly lowest level of cryoprotectant is determined), the product can be mixed with cryoprotectant to the level determined and subjected to preservation based on the temperature reduction rate determined. A safety factor, e.g. 10%, may be employed in practice for each of the cryoprotectant level and temperature reduction rate.

Refrigeration System

FIG. 14 is a piping and instrumentation diagram of the refrigeration system that continuously cools the heat exchange fluid. The refrigeration system includes a heat exchanger for exchanging heat between the heat transfer fluid and the refrigerant, which can be, for example, R404A.

1. An apparatus for preserving biological products comprising an inner housing arranged within an outer insulated housing, wherein walls of the inner housing define a compartment for receiving biological products, said walls comprising an inlet wall for inflow of a heat exchange fluid into the compartment, an opposed outlet wall for outflow of a heat exchange fluid out of the compartment, side walls and a base, the side walls and base adjoining the inlet wall to the outlet wall, wherein the inlet wall and outlet wall each include a series of apertures to accommodate a continuous heat exchange fluid flow through the apparatus such that, in operation, biological products received in the compartment of the inner housing are immersed in the heat exchange fluid to exchange heat with the heat exchange fluid.

2. The apparatus of claim 1, wherein the base includes a series of apertures.

3. The apparatus of claim 1, the apparatus including a structure receivable in the compartment for holding the biological products, wherein the structure is one or more of a tray, a rack and a basket.

4. The apparatus of claim 3, wherein the structure is suspended from a lid of the apparatus.

5. The apparatus of claim 1, wherein the outer housing comprises:
an inlet side corresponding to the inlet wall of the inner housing and defining an inlet space between the inlet side and the inlet wall; and
an outlet side corresponding to the outlet wall of the inner housing and defining an outlet space between the outlet side and the outlet wall,
wherein the inlet side includes at least one inlet communicating from an outside of the outer housing into the inlet space and the outlet side includes at least one outlet communicating from the outlet space to an outside of the outer housing, and
wherein, in operation, said heat exchange fluid is introduced into the apparatus via said at least one inlet and removed from the apparatus via said at least one outlet.

6. The apparatus of claim 5, wherein the inlet space and the outlet space are fluidly connected.

7. The apparatus of claim 5, wherein the at least one inlet and the at least one outlet are 80 mm in diameter.

8. The apparatus of claim 1, wherein, in use, the apparatus is connected to an external refrigeration system whereby the heat exchange fluid exchanges heat with a refrigerant.

9. A method of determining an amount of cryoprotectant to be added to a biological product prior to preservation, comprising:
a. determining the total surface area of an approximated geometry of the biological product, including an initial amount of cryoprotectant, to be preserved, wherein the biological product, cryoprotectant and any packaging define a sample;
b. estimating thermal properties of the sample;
c. performing computational fluid dynamics analysis on the sample within the apparatus of claim 1 based on flow constraints including any one or more of: an approximated geometry of the sample; thermal properties of the sample; the apparatus geometry; predetermined arrangement of sample in the apparatus; a predetermined inlet temperature of heat exchange fluid; and a predetermined increase in temperature of the heat exchange fluid from inlet to outlet;
d. determining an average temperature reduction rate of the core of the sample at a predetermined sample surface temperature and corresponding heat exchange fluid flow rate to obtain the average temperature reduction rate; and
e. if the fluid flow rate calculated at step (d) corresponds to a pump duty of the apparatus that is below a predetermined pump duty, selecting an amount of cryoprotectant that is a predetermined amount less than the initial amount to define a new initial amount and, if the fluid flow rate calculated at step (d) corresponds to a pump duty that is equal to a predetermined pump duty, selecting the initial amount of cryoprotectant as said amount of cryoprotectant to be added to a biological product prior to preservation; and
f. if the fluid flow rate calculated at step (d) corresponds to a pump duty that is below a predetermined pump duty, repeating steps (a) to (e) until the fluid flow rate calculated at step (d) corresponds to a pump duty that is equal to a predetermined pump duty.

10. A method of determining an amount of cryoprotectant to be added to a biological product prior to preservation, comprising:
a. determining the total surface area of an approximated geometry of the biological product, including an initial amount of cryoprotectant, to be preserved, wherein the biological product, cryoprotectant and any packaging define a sample;
b. estimating thermal properties of the sample;
c. performing computational fluid dynamics analysis on the sample within the apparatus of claim 1 based on flow constraints including any one or more of: an approximated geometry of the sample; thermal properties of the sample; the apparatus geometry; predetermined arrangement of sample in the apparatus; and a predetermined increase in temperature of the heat exchange fluid from inlet to outlet;
d. determining an average temperature reduction rate of the core of the sample at a predetermined sample surface temperature and corresponding inlet temperature of heat exchange fluid to obtain the average temperature reduction rate, and
e. if the inlet temperature of heat exchange fluid determined at step (d) corresponds to an evaporator duty of the apparatus that is below a predetermined evaporator duty, selecting an amount of cryoprotectant that is a predetermined amount less than the initial amount to define a new initial amount, and, if the fluid flow rate calculated at step (d) corresponds to a pump duty that is equal to a predetermined pump duty, selecting the initial amount of cryoprotectant as said amount of cryoprotectant to be added to a biological product prior to preservation; and
f. if the fluid flow rate calculated at step (d) corresponds to an evaporator duty that is below a predetermined evaporator duty, repeating steps (a) to (e) until the fluid flow rate calculated at step (d) corresponds to a pump duty that is equal to a predetermined pump duty.

11. The method of claim 9, wherein the initial amount of cryoprotectant is given as at least one of a wt/vol %, a wt/wt %, and a vol/vol % of the sample and wherein the step of selecting an amount of cryoprotectant that is a predetermined amount less than the initial amount involves selecting an amount of cryoprotectant that is about 1% less than the initial amount.

12.-13. (canceled)

14. Use of the apparatus of claim 1 to preserve a biological product.

15. Use of the apparatus of claim 1 to preserve a biological product, wherein the biological product contains about 0% to about 40% wt/vol of cryoprotectant.

16. Use of the apparatus of claim 1 to preserve a biological product, wherein the biological product contains about 40% wt/vol of cryoprotectant.

17. Use of the apparatus of claim 1 to preserve a biological product, wherein the biological product contains about 20% wt/vol of cryoprotectant.

18. Use of the apparatus of claim 1 to preserve a biological product, wherein the biological product contains about 0% wt/vol of cryoprotectant.

19. (canceled)

20. The method of claim 9, wherein the predetermined pump duty includes a safety factor.

21. The method of claim 10, wherein the predetermined evaporator duty includes a safety factor.

22. The method of claim 20, wherein the safety factor is about 10%.
